(19) United States
(12) Patent Application Publication
(10) Pub. No.: US 2005/0203576 A1
(43) Pub. Date: Sep. 15, 2005

(54) Surgical thread "aptos for cosmetic surgery

(76) Inventors: Marlen Andreevich Sulamanidze, MOSCOW (RU); Georgith Marlenvich Sulamanidze, Moscow (RU)

Correspondence Address: ROTHWELL, FIGG, ERNST & MANBECK, P.C., 1425 K STREET, N.W., SUITE 800, WASHINGTON, DC, 20005, US

(21) Appl. No.: 10/517,169

(22) PCT Filed: Jun. 7, 2002
(86) PCT No.: PCT/RU02/00285

§ 371 Date: Dec. 7, 2004

(51) Int. Cl. A61L 17/00

(52) U.S. Cl. 606/228

(57) ABSTRACT

A surgical thread for cosmetic surgery is provided with serially arranged inclined-notched protrusions (2) in the form of conical barbs (3) having flexible and elastic sharpened ends (4b). The barbs are arranged on one or several sides along the section of the thread (for example in an offset manner) and with a sequential alternation of barb inclinations. The thread is not fixed to a surgical needle and is introduced into soft tissues through the hole of an injection needle. The notched protrusions preserve the rupture strength of the thread and meet requirements of the functional use thereof, thereby making it possible to carry out a cosmetic surgery.

### CROSS-REFERENCE TO RELATED APPLICATION

This application is National Phase of International Application Serial No. PCT/RU02/00285, filed Jun. 7, 2002.

### FIELD OF THE INVENTION

The invention refers to medicine, to methods and techniques of carrying out surgical and plastic operations. In particular, the invention refers to medical materials used in plastic surgery during cosmetic operations.

### BRIEF DESCRIPTION OF THE BACKGROUND ART

Surgical threads for cosmetic operations are known (Ya. Zoltan, “Surgical technique and conditions for optimal wound healing”, “Meditsina”, Budapest, 1977).

Threads are made of various materials: metallic, polymeric, biological. The basic characteristics of threads are as follows: strength, smooth surface, density, biological inertness. Threads must be well tied into knots during suturing a wound, and should not loosen up.

Depending on the type of the operation, various surgical threads are used (International Conference “Present-day approaches to development of effective dressing and suturing materials and polymeric implants”, Moscow, 1995, Institute of Surgery named after A. V. Vishnevskii). These are the threads made of tantalum, golden, silver, nickel and other wires. Non-metallic threads are usually made of Dacron, nylon, kapron, polypropylene, vicryl, polysorb, etc. Monolithic, plaited, twisted threads etc. are used.

Basic characteristics of threads: strength, smooth surface, density, biologically inert nature. Also used are threads with angled protrusions providing unidirectional penetrability through tissues (patent DE 1810800 dated Jun. 4, 1970 A614 17/00). They are used for suturing of wounds and various viscera in order to accelerate the manipulation, to decrease the operational wound, and to obtain a barely visible scar.

Correction of facial wrinkles with decreased and lost skin elasticity, soft-tissue ptosis (sagging) remains one of the main problems of cosmetic and aesthetic surgery. Using the above-mentioned surgical threads does not allow to obtain the desired aesthetic effect. Their disadvantage is in that they can fix soft tissues in the existing position, however they do not provide a lifting effect, therefore their use is inefficient for flabby and sagging soft tissues.

The closest by the essence of the claimed invention are the surgical thread (patent DE 1810800 A61L 17/00, Apr. 6, 1970), a surgical thread (patent WO 98/52473, A61B 17/04, 26 Nov. 1998) used for closure of open wounds in surgical operations, and a surgical thread for cosmetic operations according to application PCT/RU99/00263.

These threads are provided with barbs of their surface, the form of the barbs being dependent on the use of these threads.

Along with it, the unidirectional positioning of the barbed protrusions on the surface of these threads does not allow to carry out the manipulation of lifting, and, accordingly, cannot provide lifting and reliable fixation of soft tissues in a new position, which is especially important for obtaining stable and long-lasting results. It is also impossible to use for these purposes the threads with barbed-protrusions with an opposite position thereof from the middle of the thread, since they are fixed to needles from one or both sides, and the thread can be pulled within tissues only to the middle of the thread.

### SUMMARY OF THE INVENTION

The task assigned is solved by the fact that the surgical thread for cosmetic operation made of a metallic (other than steel), polymeric, or biological material comprises successively positioned along its length inclined notched protrusions which, according to the invention, are made in the form of conical barbs with pointed, flexibly elastic ends with the barbs being positioned on one or several sides of the thread's section (e.g., in a staggered order) with successive alternation of the barbs' inclination. This thread is not fixed to a surgical needle, but is inserted into soft tissues through the orifice of an injection needle.

The direction and position of the notched protrusions provides, on inserting the thread into soft tissues, fixation of the thread and, accordingly, soft tissues.

It is advisable that the ends of the notched protrusions do not elevate above the thread less than the thread's diameter, the interval between the protrusions amounting to not more than two diameters of the thread.

As a variant, the thread may be provided with barbs on its opposite sides, with the directions of their inclination being opposite, as well.

Another variant is a thread provided with notched protrusions on both sides with alternating directions of their inclination.

A third variant is a thread on which the barbs formed from one or several sides are directed oppositely to each other from the centre of the thread.

Yet another variant is a thread on which the notched protrusions are made in the form of needles with sockets fixed on the thread with the alternating direction of the needles' inclination.

### BRIEF DESCRIPTION OF THE DRAWINGS

Hereinafter, the proposed invention is explained by specific examples of its realization, and the respective drawings showing the following:

FIG. 1 shows a variant of a surgical thread with alternating notched protrusions provided on the four sides along its section.

FIG. 2 shows a variant of a surgical thread with inclined notched protrusions provided on two sides of the thread.

FIG. 3 depicts a variant of surgical thread with an alternating inclination of the notched protrusions provided on two opposite sides along its section.

FIG. 4 shows a variant of a surgical thread with alternating inclined protrusions made as needles with sockets.

FIG. 5 shows a variant of surgical thread having barbs made on two sides and directed oppositely to each other from the middle of the thread.

FIG. 6 illustrates a method of subcutaneous insertion of the thread with the help of an puncture needle.

FIG. 7 shows the position of the surgical thread when the facial soft tissues are lifted.

FIG. 8 shows a lateral cut of tissues and fixation thereof by means of the surgical thread.

### DETAILED DESCRIPTION OF PREFERRED EMBODIMENTS

Surgical thread 1 for cosmetic operations with sequentially arranged along its length inclined notched protrusions 2 in the form of barbs 3 with pointed flexible and elastic ends 4.

In FIG. 1 the barbs are positioned on the four sides with regard to the section of the thread in a staggered order with successively alternating inclination of the barbs 3; 5.

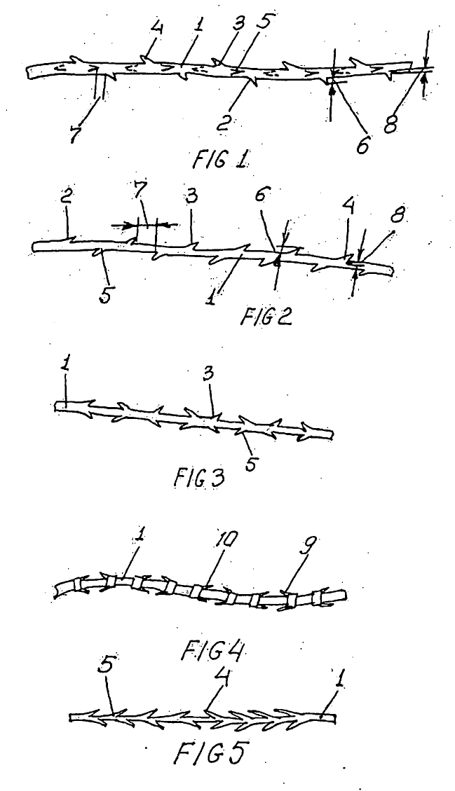

Elevation 6 of the ends 4 of the barbs above the thread should be not less than its diameter, and the interval 7 should be not less than two diameters of the thread. When providing the notched barbs 3; 5 shown in FIGS. 1, 2, 3 and 5, it is preferable to make the size of the base 8 of the notch equal to ⅓ of the thread's diameter.

In FIG. 4 the inclined notched protrusions of the thread are made in the form of needles 9 with sockets 10 for their fixation on the thread. All the mentioned parameters of the notched protrusions and their form are dictated by the necessity of preserving the rupture strength of the thread and providing due flexibility/elasticity of the protrusions' ends for good engagement and fixation of subcutaneous tissues.

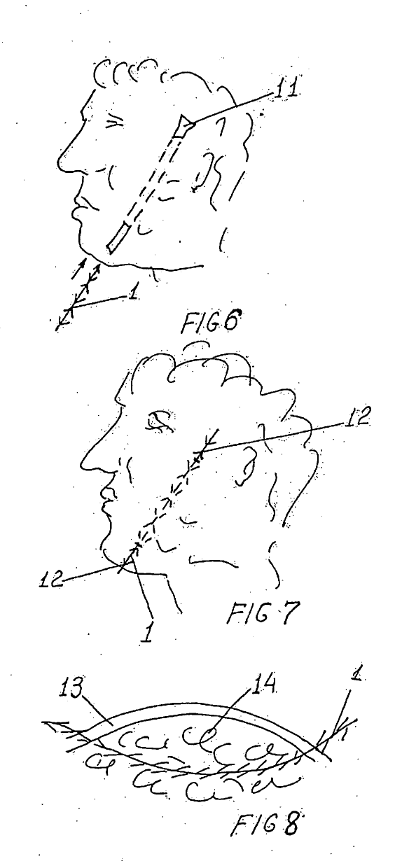

FIG. 6 shows a scheme of pulling the thread under a flabby droopy region of soft tissues of an ageing face for the purpose of its lifting. A puncture needle 11 is used to pierce the skin of the temporal portion, then it is moved subcutaneously over the previously marked contour, and is brought to the surface in the region of the nasolabial fold. Through the needle's orifice, the barbed thread is inserted, to impart the skin and soft tissues a lifted position.

FIG. 7 shows a scheme wherein the needle is withdrawn, while the thread is left, maintaining a lifted position of the tissues, since the notches prevent them from slipping down, the ends of the thread 12 extending beyond the surface of the skin.

Then, the thread's ends are cut off from the both sides near the skin surface, and are submerged subcutaneously. In this case the whole region of the ptosed soft tissues is uniformly lifted, thus smoothing out the wrinkles and skin folds.

FIG. 8 shows a longitudinal cut of a soft-tissue region—that of skin 13 and subcutaneous fat 14, wherein the thread is fixed.

Several such threads pulled subcutaneously through the necessary regions of the ageing face, provide a rejuvenating effect. When the threads with a lifting effect are used, there is no need in carrying out surgically severe operations such as rhitidoplasty, or liposuction.

Manipulations using such threads are simple and bloodless, with no scars left. They can be carried out in outpatient conditions under local anaesthesia, and with a short rehabilitation period.

### PRACTICAL APPLICABILITY

The invention can be widely used, as indicated, in various cosmetic operations. Besides, it may also be used in other surgical operations.

1. Surgical thread (1) for cosmetic operations made of a metallic, polymeric, or biological material, containing successively arranged along its length inclined notched protrusions, characterized in that the notched protrusions (2) are made in the form of conical barbs (3) with pointed flexible and elastic ends (4), the barbs (3; 5) positioned on one or several sides along the section of the thread with a successively alternating inclination thereof.

2. A surgical thread (1) for cosmetic operations, according to claim 1, characterized in that the elevation (6) of the ends (4) of the notched protrusions (2) above the thread amounts to not less than the diameter thereof, the interval (7) between the protrusions being less than two diameters of the thread.

3. A surgical thread (1) for cosmetic operations according to claim 1, characterized in that the notched protrusions (2) of the thread are provided on two opposite sides, the direction of their inclination being opposite as well.

4. A surgical thread (1) for cosmetic operations according to claim 1, characterized in that the notched protrusions are provided on two sides with an alternating direction of their inclination.

5. A surgical thread (1) for cosmetic operations according to claim 1, characterized in that the notched protrusions provided on two sides are directed oppositely to each other from the centre of the thread.

6. A surgical thread (1) for cosmetic operations according to claim 1, characterized in that the notched protrusions are provided in a form of needles (9) with sockets (10) for fixation thereof on the thread with alternating directions of the inclination of the needles.

7. The surgical thread of claim 1, wherein the barbs are positioned in a staggered order along the thread.
